(19) United States
(12) Patent Application Publication
(10) Pub. No.: US 2019/0117628 A1
(43) Pub. Date: Apr. 25, 2019

(54) Ifetroban Treatment for Systemic Sclerosis

(71) Applicants: Cumberland Pharmaceuticals Inc., Nashville, TN (US); Vanderbilt University, Nashville, TN (US)

(72) Inventors: Leo PAVLIV, Cary, NC (US); James West, Nashville, TN (US); Ines Macias-Perez, Mt. Juliet, TN (US); Erica Carrier, Nasville, TN (US)

(21) Appl. No.: 16/221,546

(22) Filed: Dec. 16, 2018

Related U.S. Application Data
(63) Continuation of application No. 15/499,523, filed on Apr. 27, 2017, now abandoned.
(60) Provisional application No. 62/328,297, filed on Apr. 27, 2016.

(51) Int. Cl. A61K 31/422; A61K 9/48; A61K 9/20; A61K 9/00

(52) U.S. Cl. CPC A61K 31/422; A61K 9/4858; A61K 47/02; A61K 9/0053; A61K 9/0019; A61K 9/2059

(57) ABSTRACT

The present invention is directed to methods of treating, preventing, and/or ameliorating systemic sclerosis, by administration of a therapeutically effective amount of ifetroban or a pharmaceutically acceptable salt thereof.

### FIELD OF THE INVENTION

The present invention is related to the use of thromboxane A₂ receptor antagonists (e.g., ifetroban) in the treatment and/or prevention of systemic sclerosis in mammals, e.g., humans, and pharmaceutical compositions for the same comprising thromboxane A₂ receptor antagonists (e.g., ifetroban) in an effective amount to treat and/or prevent these diseases.

### BACKGROUND OF THE INVENTION

Systemic sclerosis (SSc), also called scleroderma, is a rare autoimmune connective tissue disease. Systemic sclerosis is a complex and heterogeneous disease that is characterized by small vessel vasculopathy, autoantibody production, and excessive collagen deposition in the skin and internal organs (Karassa 2008). Systemic sclerosis is divided into two broad categories: limited and diffuse cutaneous disease (dcSSc) (LeRoy 1988). Limited cutaneous disease (limited scleroderma or lcSSc) is characterized by thickening of the skin confined to the area distal to the elbows and knees. The limited form of scleroderma tends to be associated with less severe internal organ involvement or systemic involvement (Barnes 2012); however, such patients may develop SSc-PAH (systemic sclerosis—pulmonary arterial hypertension) with profound effects on morbidity and mortality. Organ fibrosis is generally limited and slow to progress (Karassa 2008). Diffuse cutaneous disease (dcSSc) on the other hand involves skin thickening proximal to the elbows and knees and is associated with more rapid and severe internal organ damage. Diffuse cutaneous disease is characterized by rapidly progressing fibrosis and atrophy of the skin, joints and tendons, skeletal muscles, and internal organs including the lungs, heart, gastrointestinal tract, and kidney (Mayes 2008).

Usually the disease starts from the skin although visceral involvement may occur prior to the cutaneous presentation, and in some cases (SSc sine sclerosis) the skin may not appear to be involved. Polyarthralgia and Raynaud's phenomenon are early and almost universal clinical manifestations (Karassa 2008) and may precede other features by months to years. Subcutaneous edema is common in the early stages but eventually the skin becomes thickened and hidebound, with loss of normal folds. Telangiectasia, pigmentation, and depigmentation are characteristic signs. Digital ulceration and subcutaneous calcification are also seen. Skin-thickness progression rate has been identified as a method of predicting significant organ involvement including cardiac disease (Parks 2014) and has been traditionally been considered as a useful marker both of current severity and future prognosis (Karassa 2008). The clinical semi-quantitative assessment of skin thickness (modified Rodnan skin score [mRSS]) is currently the gold standard and the main outcome measure used in clinical trials of SSc disease-modifying agents (Castro 2010).

Pulmonary disease has emerged as the major cause of death in scleroderma patients (Black 2005). In SSc, the two most common types of direct pulmonary involvement are interstitial lung disease (ILD) and pulmonary hypertension (PH), which together account for 60% of all SSc related deaths. ILD is common in SSc. In early autopsy studies, up to 100% of patients were found to have parenchymal involvement. Parenchymal lung involvement appears early after the diagnosis of SSc, with 25% of patients developing clinically significant lung disease within 3 years. Patients with SSc can develop PH caused by pulmonary arterial hypertension (PAH), left ventricular disease or pulmonary fibrosis (Sweiss). PH can occur in all forms of SSc and is associated with early mortality. The presence of PAH is variable (Solomon 2013) and is more common in patients with lcSSc (Sweiss). Pulmonary arterial hypertension is initially silent, and early symptoms can be nonspecific. Dyspnea is a later symptom and can be attributed to multiple factors. Pulmonary arterial hypertension in SSc typically develops late in the course of patients with lcSSC (Mayes 2008). Left untreated, between 45 to 60% of the patients with PAH of any cause will die within 2 years of diagnosis. When it occurs as a manifestation of scleroderma, PAH is particularly severe and 1-year survival following diagnosis is approximately 55% (Black 2005), although survival seems to have improved since the introduction of current PAH-specific therapies such as prostanoids, endothelin receptor antagonists and phosphodiesterase type 5 inhibitors.

At present, there are no specific diagnostic tests for SSc. However, it is well recognized that the presence of specific autoantibodies is one of the most common manifestation of SSc and greater than 90% of SSc patients harbor antinuclear antibodies in their serum. Some of these are highly specific for SSc, including anti-Scl-70 and anti-centromere antibodies. Anti-Scl-70 antibodies are directed against DNA topoisomerase I and are almost exclusively present in the sera of patients with dcSSc. Anti-Scl-70 antibodies also correlate with the development of severe interstitial lung disease. Anti-centromere antibodies, on the other hand, are associated with the presence of lcSSc and the propensity to develop PAH.

Cytokine, chemokine, and growth factor alterations have been found in higher levels in both dcSSc and lcSSc. Numerous other cytokines, chemokines and regulatory proteins that are considered important participants in the immune activation of SSc have been suggested as potential biomarkers, including cluster of differentiation 40 (CD-40), chemokine ligand 2 (CCL-2), interleukin 15 (IL-15), interleukin 23 (IL-23), B-cell activating factor (BAFF), FAS receptor (FasR), and others. Elevated cytokines have been also been reported in SSc patients with anti-Scl-70 auto-antibodies (Castro 2008).

Recently, endothelial-1 (ET-1), a potent vasoconstrictor, has been shown to be significantly elevated in SSc patients with PAH and with anti-centromere antibodies. Furthermore, there was a positive correlation between these levels and systolic pulmonary pressure. N-terminal probrain natriuretic peptide (NT-pro-BNP), although not specific for PAH, reflects the myocardial response to various stimuli, such as mechanical stretch or hypoxia. ET-1 also plays a role in vasculopathy and smooth muscle cell proliferation. ET-1 levels were found to be elevated in SSs patients and to increase following exposure to cold and the triggering of the Raynaud's phenomenon. Elevated ET-1 levels also correlated with other indicators of endothelial cell activation, such as increased levels of von Willebrand factor, as well as with the levels of other endothelial cell proteins, such as thrombomodulin and adhesion molecules, including soluble Intercellular Adhesion Molecule 1 (ICAM-1) and soluble Vascular Cell Adhesion Molecule-1 (VCAM-1). Elevated expression of ET-1 and ET receptors in pulmonary parenchyma are present at early stages of development of interstitial lung disease and fibrosing alveolitis of SSc. Increased expression of Endothelial Leukocyte Adhesion Molecule-1 (ELAM-1), ICAM-1, and VCAM-1 have been found in affected skin from SSc patients and may participate in the early stages of tissue fibrosis (Castro 2008).

Transforming growth factor beta (TGF-β) is known to stimulate the synthesis and production of numerous extracellular matrix molecules involved in tissue fibrosis. TGF-β stimulates connective tissue growth factor (CTGF) synthesis in fibroblasts, vascular smooth muscle cells and endothelial cells. CTGF maintains a continuous and prolonged cycle of excessive scarring and fibrosis; thus TGF-β and CTGF may reflect activity of the fibrotic process (Castro 2008). The central role of TGF-β in inducing endothelial damage and fibroblast activation has led investigators to target this molecule as a promising site for future therapies.

To date, no therapy has been shown to modify the overall disease progression of SSc; therefore the development of a potential disease-modifying therapy in patients with SSc would address a significant unmet medical need.

### SUMMARY OF THE INVENTION

It is an object of the present invention to provide new methods of preventing and/or treating systemic sclerosis in mammals, e.g., humans.

It is an object of the present invention to provide a composition and method for reducing the effects of systemic sclerosis in mammals, e.g., humans.

It is an object of the present invention to modify the progression of myocardial fibrosis in patients with dcSSc, lcSSc, and/or SSc-PAH (e.g., as compared to placebo and, e.g., as determined by cardiac magnetic resonance imaging (MRI)).

It is an object of the present invention to improve right ventricular function in patients with dcSSc, lcSSc, and/or SSc-PAH (e.g., as compared to placebo and, e.g., as determined by echocardiography) via treatment with a thromboxane A₂ receptor antagonist (e.g., ifetroban).

It is an object of the present invention to reduce skin and peripheral vascular disease in patients with dcSSc, lcSSc, and/or SSc-PAH (e.g., as compared to placebo and, e.g., as measured by digital ulcer imaging, active digital-tip ulcer count, patient reported outcome (VAS) and the modified Rodnan skin thickness score) via treatment with a thromboxane A₂ receptor antagonist (e.g., ifetroban).

It is an object of the present invention to improve pulmonary function in patients with dcSSc, lcSSc, and/or SSc-PAH (e.g., as compared to placebo and, e.g., as measured by spirometry and diffusion capacity for carbon monoxide (DL_{CO})) via treatment with a thromboxane A₂ receptor antagonist (e.g., ifetroban).

It is an object of the present invention to reduce laboratory and physical evidence of inflammation in patients with dcSSc, lcSSc, and/or SSc-PAH (e.g., as compared to placebo as measured by serum biomarkers, erythrocyte sedimentation rate and physical examination).

It is an object of the present invention to improve quality of life in patients with dcSSc, lcSSc, and/or SSc-PAH (e.g., compared to placebo and, e.g., as measured by the patient completed Quality of Life and Scleroderma Health Assessment Questionnaires).

In accordance with the above objects, the present invention provides compositions and methods for preventing, reversing, ameliorating or treating systemic sclerosis by administering a therapeutically effective amount of a thromboxane A₂ receptor antagonist (e.g., ifetroban or a pharmaceutically acceptable salt thereof (e.g., ifetroban sodium)) to a patient in need thereof.

In accordance with the above objects, the present invention provides for methods of preventing, reversing, ameliorating or treating systemic sclerosis by administering a therapeutically effective amount of a thromboxane A₂ receptor antagonist (e.g., ifetroban) to a patient in need thereof.

In certain preferred embodiments, the mammal is a human patient with dcSSc, lcSSc, and/or SSc-PAH and the therapeutically effective amount of the thromboxane A₂ receptor antagonist or a pharmaceutically acceptable salt thereof has an action selected from the group consisting of slowing the progression of systemic sclerosis in the human patient as determined by cardiac magnetic resonance imaging (MRI); improving the exercise capacity in the human patient as determined by the six-minute walk test (6MWT); modifying the progression of myocardial fibrosis in the patient as determined by cardiac magnetic resonance imaging (MRI); improving right ventricular function in the patient as determined by echocardiography; reducing skin and peripheral vascular disease in the patient compared to placebo as measured by a test selected from the group consisting of digital ulcer imaging, active digital-tip ulcer count, patient reported outcome (VAS), the modified Rodnan skin thickness score, and any combination thereof; improving quality of life in the patient with dcSSc, lcSSc, and/or SSc-PAH compared to placebo as measured by the patient completed Quality of Life and Scleroderma Health Assessment Questionnaires; improving pulmonary function in the patient as measured by spirometry and diffusion capacity for carbon monoxide (DL_{CO}); improving laboratory and physical evidence of inflammation in the compared to placebo as measured by serum biomarkers, erythrocyte sedimentation rate, physical examination, or any combination thereof; and combinations of any of the foregoing.

In certain embodiments, the present invention is directed to a method of treating and/or ameliorating systemic sclerosis, comprising administering to a patient in need thereof a therapeutically effective amount of a thromboxane A₂ receptor antagonist to provide a desired plasma concentration of the thromboxane A₂ receptor antagonist (and/or its active metabolites) of about 0.1 ng/ml to about 100,000 ng/ml. In certain embodiments, the therapeutically effective amount of a thromboxane A2 receptor antagonist to provide a desired plasma concentration of the thromboxane A2 receptor antagonist of about 0.1 ng/ml to about 10,000 ng/ml. In some embodiments, the afore-mentioned plasma concentration is a plasma concentration at steady state. In some embodiments, the afore-mentioned plasma concentration is a maximum plasma concentration (Cmax). In certain preferred embodiments, the thromboxane A2 receptor antagonist is ifetroban or a pharmaceutically acceptable salt thereof, e.g., ifetroban sodium.

In certain embodiments, the thromboxane A₂ receptor antagonist comprises a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid (Ifetroban), and pharmaceutically acceptable salts thereof.

The invention is further directed to a method of treating dcScc (systemic sclerosis) or lcSSc (limited scleroderma) in a mammal in need of treatment thereof, comprising administering a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid (ifetroban), or a pharmaceutically acceptable salt thereof to the mammal. In certain embodiments, the thromboxane A₂ receptor antagonist comprises a therapeutically effective amount of [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid, monosodium salt (Ifetroban Sodium). In certain preferred embodiments, the mammal is a human patient. In certain preferred embodiments, the therapeutically effective amount of ifetroban slows the progression of systemic sclerosis in the human patient and/or improves the exercise capacity in the human patient and/or improves pulmonary function and/or quality of life in human patients suffering from systemic sclerosis. Certain embodiments of the invention are directed to the method, wherein lcSSc patients are treated with a therapeutically effective amount of ifetroban or a pharmaceutically acceptable salt thereof to prevent PAH from starting or progressing, or slowing its progression.

In any of the methods described above and others described herein, the ifetroban is preferably administered in an amount effective to provide a plasma concentration of the ifetroban (and/or active metabolites of ifetroban) of about 1 ng/ml to about 100,000 ng/ml or of about 1 ng/ml to about 10,000 ng/ml for ifetroban itself, and in some embodiments from about 1 ng/ml to about 1,000 ng/ml. In some embodiments, the afore-mentioned plasma concentration is a plasma concentration at steady state. In some embodiments, the afore-mentioned plasma concentration is a maximum plasma concentration (Cmax). In certain preferred embodiments where the mammal is a human patient, the therapeutically effective amount is from about 100 mg to about 2000 mg per day, or from about 10 mg or about 100 mg to about 1000 mg per day, and certain embodiments more preferably from about 50 to about 500 mg per day, or from about 100 mg to about 500 mg per day. The daily dose may be administered in divided doses or in one bolus or unit dose or in multiple dosages administered concurrently. In this regard, the ifetroban may be administered orally, intranasally, rectally, vaginally, sublingually, buccally, parenterally, or transdermally.

In certain preferred embodiments, the pharmaceutical composition described above, the therapeutically effective amount is from about 10 mg to about 1000 mg ifetroban (or pharmaceutically acceptable salt thereof) per day. In certain preferred embodiments, the therapeutically effective amount is from about 100 to about 500 mg per day, and in certain embodiments from about 150 mg to about 350 mg per day.

The present invention also relates to methods and compositions for treating systemic sclerosis in a subject(s) or patient(s) in need of treatment thereof, particularly, systemic sclerosis, the method comprising administering a therapeutically effective amount of a thromboxane A₂ receptor antagonist to a subject(s) or patient(s) in need thereof. In particular, it relates to a method of treating or preventing a disorder that results in systemic sclerosis, in a subject(s) or patient(s) in need of such treatment, comprising administering a composition comprising administering a therapeutically effective amount of a thromboxane A₂ receptor antagonist to a patient in need thereof in an amount effective to reduce the rate of systemic sclerosis. Further provided is a method of preventing systemic sclerosis in a subject(s) or patient(s) in need of such treatment, comprising administering a composition comprising a thromboxane A₂ receptor antagonist in an amount effective to reduce the formation of sclerotic tissue that would occur in the absence of such treatment.

The invention is further directed to a pharmaceutical composition comprising a thromboxane A₂ receptor antagonist or a pharmaceutically acceptable salt thereof, the thromboxane A₂ receptor antagonist being in an amount effective to treat a human patient with dcSSc, lcSSc, and/or SSc-PAH. In certain preferred embodiments, the thromboxane A₂ receptor antagonist is ifetroban or a pharmaceutically acceptable salt thereof. In certain preferred embodiments, the ifetroban salt is ifetroban sodium. In certain preferred embodiments, the therapeutically effective amount is from about 10 mg to about 1000 mg per day, and in certain embodiments from about 150 mg to about 350 mg. In certain preferred embodiments, the pharmaceutical composition is an oral solid dosage form.

The phrase “therapeutically effective amount” refers to that amount of a substance that produces some desired local or systemic effect at a reasonable benefit/risk ratio applicable to any treatment. The effective amount of such substance will vary depending upon the subject and disease condition being treated, the weight and age of the subject, the severity of the disease condition, the manner of administration and the like, which can readily be determined by one of ordinary skill in the art.

The term “thromboxane A2 receptor antagonist” as used herein refers to a compound that inhibits the expression or activity of a thromboxane receptor by at least or at least about 30%, 35%, 40%, 45%, 50%, 55%, 60%, 65%, 70%, 75%, 80%, 85%, 90%, 95%, 96%, 97%, 98%, 99%, or 100% in a standard bioassay or in vivo or when used in a therapeutically effective dose. In certain embodiments, a thromboxane A2 receptor antagonist inhibits binding of thromboxane A₂ to the receptor. Thromboxane A2 receptor antagonists include competitive antagonists (i.e., antagonists that compete with an agonist for the receptor) and non-competitive antagonists. Thromboxane A2 receptor antagonists include antibodies to the receptor. The antibodies may be monoclonal. They may be human or humanized antibodies. Thromboxane A2 receptor antagonists also include thromboxane synthase inhibitors, as well as compounds that have both thromboxane A2 receptor antagonist activity and thromboxane synthase inhibitor activity.

As used herein, the term “unit dose” refers to physically discrete units suitable as unitary dosages for mammalian subjects, each unit containing as the active ingredient a predetermined quantity of the thromboxane A2 receptor antagonist.

The term “comprising” is an inclusive term interpreted to mean containing, embracing, covering or including the elements listed following the term, but not excluding other unrecited elements.

A “therapeutically effective amount” means the amount that, when administered to an animal for treating a disease, is sufficient to effect treatment for that disease.

As used herein, the term “treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The term “parenteral” as used herein, includes subcutaneous injections, intravenous, intramuscular, intrasternal injection or infusion techniques.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

### DETAILED DESCRIPTION OF THE INVENTION

In accordance with the above stated objects, it is believed that administration of a therapeutically effective amount of a thromboxane A₂ receptor antagonist to a subject(s) or patient(s) in need thereof can prevent and/or treat systemic sclerosis.

### Thromboxane A₂ Receptor Antagonist

The discovery and development of thromboxane A₂ receptor antagonists has been an objective of many pharmaceutical companies for approximately 30 years (see, Dogne J-M, et al., Exp. Opin. Ther. Patents 11: 1663-1675 (2001)). Certain individual compounds identified by these companies, either with or without concomitant thromboxane A₂ synthase inhibitory activity, include ifetroban (BMS), ridogrel (Janssen), terbogrel (BI), UK-147535 (Pfizer), GR 32191 (Glaxo), and S-18886 (Servier). Preclinical pharmacology has established that this class of compounds has effective antithrombotic activity obtained by inhibition of the thromboxane pathway. These compounds also prevent vasoconstriction induced by thromboxane A₂ and other prostanoids that act on the thromboxane A₂ receptor within the vascular bed, and thus may be beneficial for use in preventing and/or treating hepatorenal syndrome and/or hepatic encephalopathy.

Suitable thromboxane A2 receptor antagonists for use in the present invention may include, for example, but are not limited to small molecules such as ifetroban (BMS; [1S-(1α,2α,3α,4α)]-2-[[3-[4-[(pentylamino)carbony-1]-2-oxazolyl]-7-oxabicyclo[2.2.1]hept-2 yl]methyl]benzenepropanoic acid), as well as others described in U.S. Patent Application Publication No. 2009/0012115, the disclosure of which is hereby incorporated by reference in its entirety.

Additional thromboxane A2 receptor antagonists suitable for use herein are also described in U.S. Pat. No. 4,839,384 (Ogletree); U.S. Pat. No. 5,066,480 (Ogletree, et al.); U.S. Pat. No. 5,100,889 (Misra, et al.); U.S. Pat. No. 5,312,818 (Rubin, et al.); U.S. Pat. No. 5,399,725 (Poss, et al.); and U.S. Pat. No. 6,509,348 (Ogletree), the disclosures of which are hereby incorporated by reference in their entireties. These may include, but are not limited to, interphenylene 7-oxabicyclo-heptyl substituted heterocyclic amide prostaglandin analogs as disclosed in U.S. Pat. No. 5,100,889, including:
[1S-(1α, 2α, 3α, 4α)]-2-[[3-[4-[[(4-cyclo-hexylbutyl)amino]carbonyl]-2-oxazolyl]-7-oxabicyclo[2.2.1]-hept-2-yl]methyl]benzenepropanoic acid (SQ 33,961), or esters or salts thereof;
[1S-(1α, 2 α, 3 α, 4α)]-2-[[3-[4-[[[(4-chloro-phenyl)-butyl]amino]carbonyl]-2-oxazolyl]-7-oxabicyclo[2.2.1]hept-2-yl]methyl]benzenepropanoic acid or esters, or salts thereof;
[1S-(1α, 2 α, 3 α, 4α)]-3-[[3-[4-[[(4-cycloh-exylbutyl)-amino]carbonyl]-2-oxazolyl]-7-oxabicyclo]2.2.1]hept-2-yl]benzene acetic acid, or esters or salts thereof;
[1S-(1α, 2 α, 3 α, 4α)]-[2-[[3-[4-[[(4-cyclohexyl-butyl)amino]carbonyl]-2-oxazolyl]-7-oxabicyclo[2.2.1]hept-2-yl]methyl]phenoxy]acetic acid, or esters or salts thereof;
[1S-(1α, 2α, 3α, 4α]-2-[[3-[4-[[(7,7-dime-thyloctyl)-amino]carbonyl]-2-oxazolyl]-7-oxabicyclo[2.2.1]hept-2-yl]-methyl]benzenepropanoic acid, or esters or salts thereof.
7-oxabicycloheptyl substituted heterocyclic amide prostaglandin analogs as disclosed in U.S. Pat. No. 5,100,889, issued Mar. 31, 1992, including [1S-[1α, 2α (Z), 3α, 4α)]-6-[3-[4-[[(4-cyclohexylbutyl)amino]-carbonyl]-2-oxazolyl]-7-oxabicyclo[2.2.1]hept-2-yl]-4-hexenoic acid, or esters or salts thereof;
[1S-[1α, 2α (Z), 3α, 4α)]]-6-[3-[4-[[(4-cyclohexyl-butyl)amino]carbonyl]-2-thiazolyl]-7-oxabicyclo[2.2.1]hept-2-yl]-4-hexenoic acid, or esters or salts thereof;
[1S-[1α, 2α (Z), 3α, 4α)]]-6-[3-[4-[[(4-cyclohexyl-butyl)methylamino]carbonyl]-2-oxazolyl]-7-oxabicyclo-[2.2.1]hept-2-yl]-4-hexenoic acid, or esters or salts thereof;
[1S-[1α, 2α (Z), 3α, 4α)]]-6-[3-[4-[(1-pyrrolidinyl)-carbonyl]-2-oxazolyl]-7-oxabicyclo[2.2.1]hept-2-yl]-4-hexenoic acid, or esters or salts thereof;
[1S-[1α, 2α (Z), 3α, 4α)]]-6-[3-[4-[(cyclohexylamino)-carbonyl]-2-oxazolyl]-7-oxabicyclo[2.2.1]hept-2-yl-4-hexenoic acid or esters or salts thereof;
[1S-[1α, 2α (Z), 3α, 4α)]]-6-[3-[4-[[(2-cyclohexyl-ethyl)amino]carbonyl]-2-oxazolyl]-7-oxabicyclo[2.2.1]hept-2-yl]-4-hexenoic acid, or esters or salts thereof;
[1S-[1α, 2α (Z), 3α, 4α)]]-6-[3-[4-[[[2-(4-chloro-phenyl)ethyl]amino]carbonyl]-2-oxazolyl]-7-oxabicyclo-[2.2.1]hept-2-yl]-4-hexenoic acid, or esters or salts thereof;
[1S-[1α, 2α (Z), 3α, 4α)]-6-[3-[4-[[(4-chlorophenyl)-amino]carbonyl]-2-oxazolyl]-7-oxabicyclo[2.2.1]hept-2-yl]-4-hexenoic acid, or esters or salts thereof;
[1S-[1α, 2α (Z), 3α, 4α)]]-6-[3-[4-[[[4-(4-chloro-phenyl)butyl]amino]carbonyl]-2-oxazolyl]-7-oxabicyclo-[2.2.1]hept-2-yl]-4-hexenoic acid, or esters or salts thereof;
[1S-[11α, 2α (Z), 3α, 4α)]]-6-[3-[4.alpha.-[[(6-cyclohexyl-hexyl)amino]carbonyl]-2-oxazolyl]-7-oxabicyclo[2.2.1]hept-2-yl]-4-hexenoic acid, or esters, or salts thereof;
[1S-[1α, 2α (Z), 3α, 4α)]]-6-[3-[4-[[(6-cyclohexyl-hexyl)amino]carbonyl]-2-oxazolyl]-7-oxabicyclo[2.2.1]hept-2-yl]-4-hexenoic acid, or esters or salts thereof;
[1S-[1α, 2α (Z), 3α, 4α]]-6-[3-[4-[(propylamino)-carbonyl]-2-oxazolyl]-7-oxabicyclo[2.2.1]hept-2-yl]-4-hexenoic acid, or esters or salts thereof
[1S-[1α, 2α (Z), 3α, 4α)]]-6-[3-[4-[[(4-butylphenyl)-amino]carbonyl]-2-oxazolyl]-7-oxabicyclo[2.2.1]hept-2-yl]-4-hexenoic acid, or esters or salts thereof;
[1S-[1α, 2α (Z), 3α, 4α)]]-6-[3-[4-[(2,3-dihydro-1H-indol-1-yl)carbonyl]-2-oxazolyl]-7-oxabicyclo[2.2.1]hept-2-yl]-4-hexenoic acid, or esters or salts thereof;
[1S-[1α, 2α (Z), 3α, 4α)]]-6-[3-[4-[[(4-cyclohexyl-butyl)amino]carbonyl]-2-oxazolyl]-7-oxabicyclo[2.2.1]hept-2-yl]-N-(phenylsulfonyl)-4-hexenamide;
[1S-[11α, 2α (Z), 3α, 4α)]]-6-[3-[4-[[(4-cyclohexyl-butyl)amino]carbonyl]-2-oxazolyl]-N-(methylsulfonyl)-7-oxabicyclo[2-.2.1]hept-2-yl]-4-hexenamide;
[1S-[1α, 2α (Z), 3α, 4α)]]-7-[3-[4-[[(4-cyclohexyl-butyl)amino]carbonyl]-2-oxazolyl]-7-oxabicyclo (2.2.1]hept-2-yl]-5-heptenoic acid, or esters or salts thereof;
[1S-[1α, 2α (Z), 3α, 4α)]]-6-[3-[4-[[(4-cyclohexyl-butyl)amino]carbonyl]-1H-imidazol-2-yl]-7-oxabicyclo-[2.2.1]hept-2-yl]-4-hexenoic acid or esters or salts thereof;
[1S-[1α, 2α, 3α, 4α)]-6-[3-[4-[[(7, 7-dimethyloctyl)-amino]carbonyl]-2-oxazolyl]-7-oxabicyclo[2.2.1]hept-2-yl]-4-hexenoic acid, or esters or salts thereof;
[1S-[1α, 2α(E), 3α, 4α)]]-6-[3-[4-[[(4-cyclohexyl-butyl)amino]carbonyl]-2-oxazolyl]-7-oxabicyclo[2.2.1]hept-2-yl]-4-hexenoic acid;
[1S-[1α, 2α, 3α, 4α)]-3-[4-[[(4-(cyclohexylbutyl)-amino]carbonyl]-2-oxazolyl]-7-oxabicyclo[2.2.1]heptane-2-hexanoic acid or esters or salts thereof,
[1S-[1α, 2α(Z), 3α, 4α)]]-6-[3-[4-[[(4-cyclohexyl-butyl)amino]carbonyl]-2-oxazolyl]-7-oxabicyclo-[2.2.1]hept-2-yl]-4-hexenoic acid, or esters or salts thereof;
7-oxabicycloheptane and 7-oxabicycloheptene compounds disclosed in U.S. Pat. No. 4,537,981 to Snitman et al, the disclosure of which is hereby incorporated by reference in its entirety, such as [1S-(1α, 2α(Z), 3α(1E, 3S*, 4R*), 4α)]]-7-[3-(3-hydroxy-4-phenyl-1-pentenyl)-7-oxabicyclo[2.2.1]hept-2-yl]-5-heptenoic acid (SQ 29,548); the 7-oxabicycloheptane substituted aminoprostaglandin analogs disclosed in U.S. Pat. No. 4,416,896 to Nakane et al, the disclosure of which is hereby incorporated by reference in its entirety, such as [1S-[1α, 2α(Z), 3α, 4α)]]-7-[3-[[2-(phenylamino)carbonyl]-hydrazino]methyl]-7-oxabicyclo[2.2.1]hept-2-yl]-5-heptenoic acid; the 7-oxabicycloheptane substituted diamide prostaglandin analogs disclosed in U.S. Pat. No. 4,663,336 to Nakane et al, the disclosure of which is hereby incorporated by reference in its entirety, such as, [1S-[1α, 2α(Z), 3α, 4α)]]-7-[3-[[[[(1-oxoheptyl)amino]-acetyl]amino]methyl]-7-oxabicyclo[2.2.1]hept-2-yl]-5-heptenoic acid and the corresponding tetrazole, and [1S-[1α, 2α(Z), 3α,4α)]]-7-[3-[[[[[(4-cyclohexyl-1-oxobutyl)-amino]acetyl]amino]methyl]-7-oxabicyclo]2.2.1]hept-2-yl]-5-heptenoic acid;
7-oxabicycloheptane imidazole prostaglandin analogs as disclosed in U.S. Pat. No. 4,977,174, the disclosure of which is hereby incorporated by reference in its entirety, such as [1S-[1α, 2α(Z), 3α, 4α)]]-6-[3-[[4-(4-cyclohexyl-1-hydroxybutyl)-1H-imidazole-1-yl]methyl]-7-oxabicyclo[2.2.1]hept-2-yl]-4-hexenoic acid or its methyl ester;
[1S-[1α, 2α(Z), 3α, 4α)]]-6-[3-[[4-(3-cyclohexyl-propyl)-1H-imidazol-1-yl]methyl]-7-oxabicyclo[2.2.1]hept-2-yl]-4-hexenoic acid or its methyl ester;
[1S-[1α, 2α(X(Z), 3α, 4α)]]-6-[3-[[4-(4-cyclohexyl-1-oxobutyl)-1H-imidazol-1-yl]methyl]-7-oxabicyclo[2.2.1]hept-2-yl]-4-hexenoic acid or its methyl ester;
[1S-[1α, 2α(Z), 3α, 4α]]-6-[3-(1H-imidazol-1-ylmethyl)-7-oxabicyclo[2.2.1]hept-2-yl]-4-hexenoic acid or its methyl ester; or
[1S-[1α, 2α(Z), 3α, 4α)]]-6-[3-[[4-[[(4-cyclohexyl-butyl)amino]carbonyl]-1H-imidazol-1-yl]methyl-7-oxabicyclo-[2.2.1]-hept-2-yl]-4-hexenoic acid, or its methyl ester;

The phenoxyalkyl carboxylic acids disclosed in U.S. Pat. No. 4,258,058 to Witte et al, the disclosure of which is hereby incorporated by reference in its entirety, including 4-[2-(benzenesulfamido)ethyl]phenoxy-acetic acid (BM 13,177-Boehringer Mannheim), the sulphonamidophenyl carboxylic acids disclosed in U.S. Pat. No. 4,443,477 to Witte et al, the disclosure of which is hereby incorporated by reference in its entirety, including 4-[2-(4-chlorobenzenesulfonamido)ethyl]-phenylacetic acid (BM 13,505, Boehringer Mannheim), the arylthioalkylphenyl carboxylic acids disclosed in U.S. Pat. No. 4,752,616, the disclosure of which is hereby incorporated by reference in its entirety, including 4-(3-((4-chlorophenyl) sulfonyl)propyl)benzene acetic acid.

Other examples of thromboxane A₂ receptor antagonists suitable for use herein include, but are not limited to vapiprost (which is a preferred example), (E)-5-[[[(pyridinyl)]3-(trifluoromethyl)phenyl]methylene]amino]-oxy]pentanoic acid also referred to as R68,070-Janssen Research Laboratories, 3-[1-(4-chlorophenylmethyl)-5-fluoro-3-methylindol-2-yl]-2,-2-dimethylpropanoic acid [(L-655240 Merck-Frosst) Eur. J. Pharmacol. 135(2):193, Mar. 17, 87], 5(Z)-7-([2,4,5-cis]-4-(2-hydroxyphenyl)-2-trifl-uoromethyl-1,3-dioxan-5-yl)heptenoic acid (ICI 185282, Brit. J. Pharmacol. 90 (Proc. Suppl):228 P-Abs, March 87), 5(Z)-7-[2,2-dimethyl-4-phenyl-1,3-dioxan-cis-5-yl]heptenoic acid (ICI 159995, Brit. J. Pharmacol. 86 (Proc. Suppl):808 P-Abs., December 85), N,N′-bis[7-(3-chlorobenzeneamino-sulfony-1)-1,2,3,4-tetrahydro-isoquinolyl]disulfonylimide (SKF 88046, Pharmacologist 25(3):116 Abs., 117 Abs, August 83), (1.alpha.(Z)-2.beta., 5.alpha.]-(+)-7-[5-[[(1,1′-biphenyl)-4-yl]-methoxy]-2-(4-morpholinyl)-3-oxocyclopentyl]-4-heptenoic acid (AH 23848-Glaxo, Circulation 72(6):1208, December 85, levallorphan allyl bromide (CM 32,191 Sanofi, Life Sci. 31 (20-21):2261, Nov. 15, 82), (Z,2-endo-3-oxo)-7-(3-acetyl-2-bicyclo [2.2.1]heptyl-5-hepta-3Z-enoic acid, 4-phenyl-thiosemicarbazone (EP092-Univ. Edinburgh, Brit. J. Pharmacol. 84(3):595, March 85); GR 32,191 (Vapiprost)-[1R-[1.alpha.(Z), 2.beta., 3.beta., 5.alpha.]]-(+)-7-[5-([1,1′-biphenyl]-4-ylmethoxy)-3-hydroxy-2-(1-piperidinyl)cyclopentyl]-4-heptenoic acid; ICI 192,605-4(Z)-6-[(2,4,5-cis)2-(2-chlorophenyl)-4-(2-hydroxyphenyl)-1,3-dioxan-5-yl]hexenoic acid; BAY u 3405 (ramatroban)-3-[[(4-fluorophenyl)-sulfonyl]amino]-1,2,3,4-tetrahydro-9H-c-arbazole-9-propanoic acid; or ONO 3708-7-[2.alpha., 4.alpha.-(dimethylmethano)-6.beta.-(2-cyclopentyl-2.beta.-hydroxyacetami-do)-1.alpha.-cyclohexyl]-5(Z)-heptenoic acid; (.+−.)(5Z)-7-[3-endo-((phenylsulfonyl)amino]-bicyclo [2.2.1]hept-2-exo-yl]-heptenoic acid (S-1452, Shionogi domitroban, Anboxan®.); (−)6,8-difluoro-9-p-methylsulfonylben-zyl-1,2,3,4-tetrahydrocarbazol-1-yl-acetic acid (L670596, Merck) and (3-[1-(4-chlorobenzyl)-5-fluoro-3-methyl-indol-2-yl]-2,2-dimethylpropanoic acid (L655240, Merck).

The preferred thromboxane A2 receptor antagonist of the present invention is ifetroban or any pharmaceutically acceptable salts thereof.

In certain preferred embodiments the preferred thromboxane A2 receptor antagonist is ifetroban sodium (known chemically as [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid, monosodium salt.

Two known pharmacological actions of ifetroban resulting from TP antagonism include inhibition of smooth muscle contractions (Ogletree, 1992) and inhibition of platelet shape change and aggregation. These divergent pharmacological actions suggest varied potential therapeutic indications. Recently, the effect of TP receptor antagonism was studied in mice with mechanical constriction of the pulmonary artery, a model of PAH-associated right ventricular hypertrophy. Treatment with ifetroban reduced right ventricular fibrosis and cardiomyocyte hypertrophy in pulmonary artery banded mice, and increased ratio of the early (E) to late (A) ventricular filling velocities (E/A ratio), one indicator of cardiac efficiency. This was associated with augmented right ventricular expression of anti-fibrotic and muscularization genes, as well as decreased expression of genes associated with inflammation.

Systemic sclerosis (SSc, scleroderma) is potential therapeutic target of ifetroban given the known role of platelet activation in the initiation and perpetuation of autoimmune inflammatory processes and therefore fibrosis. By antagonizing the TP receptor, ifetroban is capable of inhibiting signals from thromboxane and F2-isoprostane not only on platelets but also on endothelium and other immune cells potentially stemming from the inflammation.

Given the preclinical work indicating that administration of ifetroban is capable of preventing cardiac fibrosis in a model of pulmonary arterial hypertension, evaluating a treatment that may delay or inhibit tissue fibrosis could provide significant improvement in quality of life for these patients. In addition, ifetroban could potentially also modify the skin disease in SSc and skin assessments will be included for all patients in this study.

The heart is a major organ involved in scleroderma and the presence of cardiac involvement in SSc is often underestimated and is a sign of poor prognosis (Champion). SSc patients, especially those with PAH, often experience a poor quality of life; many with severe disease are often unable to perform even simple routine standard of care daily activities without severe shortness of breath, fatigue and fainting, and because they experience a risk of early death due to the rapid deterioration of their pulmonary and cardiac systems, the need for effective treatment is important. This study will provide safety and initial efficacy data for ifetroban in patients with dcSSc, lcSSc, and SSc-PAH.

### Methods of Treatment

In certain embodiments of the present invention there is provided a method of preventing and/or treating and/or ameliorating systemic sclerosis in one or more organs or tissues in a patient or patient population by administration of a therapeutically effective amount of a thromboxane A₂ receptor antagonist to a patient(s) in need thereof.

The administration of a therapeutically effective amount of a thromboxane A₂ receptor antagonist may be accomplished via any therapeutically useful route of administration, including but not limited to orally, intranasally, rectally, vaginally, sublingually, buccally, parenterally, or transdermally. In certain preferred embodiments, the thromboxane A₂ receptor antagonist is administered parenterally. In certain further embodiments, the thromboxane A₂ receptor antagonist is administered by intra-articular injection. In certain further embodiments, the thromboxane A₂ receptor antagonist is administered directly to the affected anatomic site. In another embodiment, the thromboxane A₂ receptor antagonist is administered through the hepatic artery.

In any of the methods described above and others described herein, the thromboxane A₂ receptor antagonist (e.g., ifetroban) is preferably administered in an amount effective to provide a plasma concentration of the thromboxane A₂ receptor antagonist (and/or active metabolites thereof) of about 1 ng/ml to about 100,000 ng/ml or of about about 0.1 ng/ml; or 1 ng/ml to about 10,000 ng/ml for ifetroban itself, and in some embodiments from about 1 ng/ml to about 1,000 ng/ml or more (e.g., in some embodiments up to about 10,000 ng/ml, and in further embodiments up to about 100,000 ng/ml). In some embodiments, the afore-mentioned plasma concentration is a plasma concentration at steady state. In some embodiments, the afore-mentioned plasma concentration is a maximum plasma concentration (Cmax). In certain preferred embodiments where the mammal is a human patient, the therapeutically effective amount is from about 100 mg to about 2000 mg per day, or from about 10 mg or about 100 mg to about 1000 mg per day, and certain embodiments more preferably from about 100 to about 500 mg per day. The daily dose may be administered in divided doses or in one bolus or unit dose or in multiple dosages administered concurrently. In this regard, the ifetroban may be administered orally, intranasally, rectally, vaginally, sublingually, buccally, parenterally, or transdermally.

In one embodiment where the mammal is a human patient, the therapeutically effect amount of ifetroban is about 250 mg daily, taken orally.

The dose administered should be adjusted according to age, weight and condition of the patient, as well as the route of administration, dosage form and regimen and the desired result.

In order to obtain the desired plasma concentration of thromboxane A₂ receptor antagonists for the treatment or prevention of systemic sclerosis, daily doses of the thromboxane A₂ receptor antagonists preferably range from about 0.1 mg to about 5000 mg. In certain preferred embodiments, the daily dose of thromboxane A₂ receptor antagonists for the treatment or prevention of systemic sclerosis may range from about 1 mg to about 2000 mg; about 10 mg to about 1000 mg; from about 100 mg to about 1000 mg; from about 50 mg to about 500 mg; about 100 mg to about 500 mg; or from about 150 mg to about 300 mg per day.

In certain preferred embodiments, a daily dose of ifetroban sodium from about 10 mg to about 500 mg, preferably from about 150 mg to about 300 mg (ifetroban free acid amounts) will produce therapeutically effective plasma levels of ifetroban free acid for the treatment or prevention of systemic sclerosis.

When the thromboxane A₂ receptor antagonist is ifetroban, the desired plasma concentration for providing an inhibitory effect of A₂/prostaglandin endoperoxide receptor (TP) activation, and thus a reduction of cerebral microvascular activation should be greater than about 10 ng/mL (ifetroban free acid). Some inhibitory effects of thromboxane A₂ receptor antagonist, e.g., ifetroban, may be seen at concentrations of greater than about 1 ng/mL.

The dose administered must be carefully adjusted according to age, weight and condition of the patient, as well as the route of administration, dosage form and regimen and the desired result.

In certain preferred embodiments where the thromboxane A₂ receptor antagonist is ifetroban or a pharmaceutically acceptable salt thereof, a daily dose of ifetroban sodium from about 10 mg to about 500 mg, preferably from about 150 mg to about 300 mg (ifetroban free acid amounts) will produce effective plasma levels of ifetroban free acid.

### Pharmaceutical Compositions

The thromboxane A₂ receptor antagonists of the present invention may be administered by any pharmaceutically effective route. For example, the thromboxane A₂ receptor antagonists may be formulated in a manner such that they can be administered orally, intranasally, rectally, vaginally, sublingually, buccally, parenterally, or transdermally, and, thus, be formulated accordingly.

In certain embodiments, the thromboxane A₂ receptor antagonists may be formulated in a pharmaceutically acceptable oral dosage form. Oral dosage forms may include, but are not limited to, oral solid dosage forms and oral liquid dosage forms.

Oral solid dosage forms may include, but are not limited to, tablets, capsules, caplets, powders, pellets, multiparticulates, beads, spheres and any combinations thereof. These oral solid dosage forms may be formulated as immediate release, controlled release, sustained (extended) release or modified release formulations.

The oral solid dosage forms of the present invention may also contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof.

Depending on the desired release profile, the oral solid dosage forms of the present invention may contain a suitable amount of controlled-release agents, extended-release agents, modified-release agents.

Oral liquid dosage forms include, but are not limited to, solutions, emulsions, suspensions, and syrups. These oral liquid dosage forms may be formulated with any pharmaceutically acceptable excipient known to those of skill in the art for the preparation of liquid dosage forms. For example, water, glycerin, simple syrup, alcohol and combinations thereof.

In certain embodiments of the present invention, the thromboxane A2 receptor antagonists may be formulated into a dosage form suitable for parenteral use. For example, the dosage form may be a lyophilized powder, a solution, suspension (e.g., depot suspension).

In other embodiments, the thromboxane A2 receptor antagonists may be formulated into a topical dosage form such as, but not limited to, a patch, a gel, a paste, a cream, an emulsion, liniment, balm, lotion, and ointment.

### DETAILED DESCRIPTION OF THE PREFERRED EMBODIMENTS

The following examples are not meant to be limiting and represent certain embodiments of the present invention.

### Example 1

In this example, ifetroban sodium capsules are prepared with the following ingredients listed in Table 1:

TABLE 1

| Ingredients | Percent by weight |
|---|---|
| Na salt of Ifetroban | 35 |
| Mannitol | 50 |
| Microcrystalline Cellulose | 8 |
| Crospovidone | 3.0 |
| Magnesium Oxide | 2.0 |
| Magnesium Stearate | 1.5 |
| Colloidal Silica | 0.3 |

The sodium salt of ifetroban, magnesium oxide, mannitol, microcrystalline cellulose, and crospovidone is mixed together for about 2 to about 10 minutes employing a suitable mixer. The resulting mixture is passed through a #12 to #40 mesh size screen. Thereafter, magnesium stearate and colloidal silica are added and mixing is continued for about 1 to about 3 minutes.

The resulting homogeneous mixture is then filled into capsules each containing 50 mg, ifetroban sodium salt.

### Example II

In this example, 1000 tablets each containing 400 mg of Ifetroban sodium are produced from the following ingredients listed in Table 2:

TABLE 2

| Ingredients | Amount | |
|---|---|---|
| Na salt of Ifetroban | 400 | gm |
| Corn Starch | 50 | g |
| Gelatin | 7.5 | g |
| Microcrystalline | 25 | g |
| Cellulose (Avicel) | | |
| Magnesium Stearate | 2.5 | g |

### Example III

In this example. An injectable solution of ifetroban sodium is prepared for intravenous use with the following ingredients listed in Tables 3a and 3b:

TABLE 3a

| Ingredients | Amount | |
|---|---|---|
| Ifetroban Sodium | 2500 | mg |
| Methyl Paraben | 5 | mg |
| Propyl Paraben | 1 | mg |
| Sodium Chloride | 25,000 | mg |
| Water for injection q.s. | 5 | liter |

TABLE 3b

| Ingredients | Amount | |
|---|---|---|
| Ifetroban Sodium | 52.5 | mg |
| Sodium Phosphate Dibasic Anhydrous | 345 | mg |
| Sodium Phosphate Monobasic Anhydrous | 1.0 | g |
| Sodium Chloride | 21.5 | g |
| Water for injection q.s. | 5 | liter |

The sodium salt of ifetroban, buffers and sodium chloride are dissolved in 3 liters of water for injection and then the volume is brought up to 5 liters. The solution is filtered through a sterile filter and aseptically filled into pre-sterilized vials which are then closed with pre-sterilized rubber closures. Each vial contains a concentration of 50 mg of active ingredient per 5 ml of solution.

### Example IV

In Example 4, a phase 2 multicenter, randomized, double-blind, placebo-controlled, study in patients with dcSSc, lcSSc or SSc-PAH. There will be a 365 day blinded treatment in this study and with assessments being performed at Screening (−14 days to Study Hour 0), Baseline (Study Visit 1/Study Hour 0), Week 12 (Study Visit 2), Week 26 (Study Visit 3), Week 39 (Study Visit 4), Week 52 (Study Visit 5), and Week 56 (Study Visit 6) is conducted. Safety will be monitored throughout the Treatment Period. Separate randomization schemes will be generated for the SSc-PAH and dcSSc patient groups allowing enrollment to progress independently between patient groups. Fourteen (14) patients with SSc-PAH will be enrolled in this study, with 10 receiving ifetroban and 4 receiving matching placebo. These patients may have lcSSc or dcSSc. Twenty (20) patients with dcSSc will be enrolled in this study, with 14 receiving ifetroban and 6 receiving matching placebo. Inclusion criteria for SSc are: Adults with SSc according to the 2013 ACR/EULAR criteria (Appendix A) and with diffuse cutaneous involvement within 5 years following initial diagnosis as defined by the onset of the first non-Raynaud symptom. Inclusion criteria for SSc-PAH are: Adults with confirmed SSc-PAH (limited or diffuse SSc); stable oral therapy for PAH for at least 30 days; and New York Heart Association Class I-III Heart Failure.

Subjects randomized to oral ifetroban will be administered five 50 mg capsules by mouth per day (250 mg daily dose) for 365 days. Subject randomized to placebo will take five matching placebo capsules by mouth per day for 365 days.

The drug product is supplied as a capsule dosage form (size #1, white opaque) for oral administration. The formulation consists of ifetroban, mannitol, microcrystalline cellulose, crospovidone, magnesium oxide, colloidal silicon dioxide, and magnesium stearate. Capsules are filled into high density polyethylene bottles and sealed with screw-cap closures. Placebo for Ifetroban capsules are formulated as a dry powder blend filled into capsules. The formulation consists of microcrystalline cellulose, crospovidone, colloidal silicon dioxide, and magnesium stearate. Capsules are filled into high density polyethylene bottles and sealed with screw-cap closures. Ifetroban and placebo capsules should be administered in a fasting state. Meals following administration should be held for at least 30 minutes following study drug administration.

The objectives of the study are as follows:

To determine the safety of ifetroban in patients with diffuse cutaneous SSc or SSc-PAH compared to placebo as measured by treatment emergent adverse events (TEAE) and standard laboratory assessments.

To determine if ifetroban can modify the progression of myocardial fibrosis in patients with diffuse SSc or SSc-PAH compared to placebo as determined by cardiac magnetic resonance imaging (MRI).

To determine if ifetroban improves right ventricular function in patients with diffuse cutaneous SSc or SSc-PAH compared to placebo as determined by echocardiography.

To determine if ifetroban improves exercise capacity in patients with diffuse cutaneous SSc or SSc-PAH compared to placebo as determined by the six-minute walk test (6MWT).

To determine if ifetroban reduces skin and peripheral vascular disease in patients with diffuse cutaneous SSc or SSc-PAH compared to placebo as measured by digital ulcer imaging, active digital-tip ulcer count, patient reported outcome (VAS) and the modified Rodnan skin thickness score.

To determine if ifetroban improves quality of life in patients with diffuse cutaneous SSc or SSc-PAH compared to placebo as measured by the patient completed Quality of Life and Scleroderma Health Assessment Questionnaires.

To determine if ifetroban improves pulmonary function in patients with diffuse cutaneous SSc or SSc-PAH compared to placebo as measured by spirometry and diffusion capacity for carbon monoxide (DLCO).

To determine if ifetroban improves laboratory and physical evidence of inflammation in patients with diffuse cutaneous SSc or SSc-PAH compared to placebo as measured by serum biomarkers, erythrocyte sedimentation rate and physical examination.

To determine if ifetroban alters biomarkers in the skin in patients with diffuse cutaneous SSc compared to placebo as measured by skin biopsy biomarkers.

### CONCLUSION

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification is to be regarded in an illustrative manner rather than a restrictive sense.

### REFERENCES

Barnes J, Mayes M D. Epidemiology of systemic sclerosis: incidence, prevalence, survival, risk factors, malignancy, and environmental triggers. Curr Opin Rheumatol. 2012 March; 24(2): 165-70.
Black C. Pulmonary arterial hypertension: are we doing enough to identify systemic sclerosis patients at high risk of this rare condition? Rheumatology (Oxford). 2005 February; 44(2):141-2.
Castro S V, Jimenez S A. Biomarkers in systemic sclerosis. Biomark Med. 2010 February; 4(1):133-47.
Champion H C. The heart in scleroderma. Rheum Dis Clin North Am. 2008 February; 34(1):181-90; viii.
Gaubitz M. Epidemiology of connective tissue disorders. Rheumatology (Oxford). 2006 October; 45 Suppl 3:iii3-4.
Karassa F B, Ioannidis J P. Mortality in systemic sclerosis. Clin Exp Rheumatol. 2008 September-October; 26(5 Suppl 51):S85-93.
Kowal-Bielecka O, Landewé R, Avouac J, Chwiesko S, et al.; EUSTAR Co-Authors. EULAR recommendations for the treatment of systemic sclerosis: a report from the EULAR Scleroderma Trials and Research group (EUSTAR). Ann Rheum Dis. 2009 May; 68(5):620-8.
Mayes M D. Primer on the Rheumatic Diseases Kippel, J H, Stone J H, Crofford Lej, White P H (Eds.) 2008, XVII, 343-350.
Ntusi N A, Piechnik S K, Francis J M, et al. Subclinical myocardial inflammation and diffuse fibrosis are common in systemic sclerosis—a clinical study using myocardial T1-mapping and extracellular volume quantification. J Cardiovasc Magn Reson. 2014 Mar. 4; 16:21.
Parks J L, Taylor M H, Parks L P, Silver R M. Systemic sclerosis and the heart. Rheum Dis Clin North Am. 2014 February; 40(1):87-102.
Rosenfeld L, Grover G J, Stier C T Jr. Ifetroban sodium: an effective TxA2/PGH2 receptor antagonist. Cardiovasc Drug Rev. 2001 Summer; 19(2):97-115.
Solomon J J, Olson A L, Fischer A, et al. Scleroderma lung disease. Eur Respir Rev. 2013 Mar. 1; 22(127):6-19.
Sweiss N, Hushaw L, Thenappan T, Sawaqed R. Diagnosis and Management of Pulmonary Hypertension in Systemic Sclerosis. Curr Rheumatol Rep 2010; 12(1):8-18.
Plastiras S C, Toumanidis S T. Systemic sclerosis: the heart of the matter. Hellenic J Cardiol. 2012 July-August; 53(4):287-300.
Ware J E Jr, Sherbourne C D. The MOS 36-item short-form health survey (SF-36). I. Conceptual framework and item selection. Med Care. 1992 June; 30(6):473-83.
FDA. Guidance for Clinical Investigators, Industry, and FDA Staff: Financial Disclosure by Clinical Investigators. February 2013. http://www.fda.gov/downloads/RegulatoryInformation/Guidances/UCM341008.pdf
Van den Hoogen F, et al. 2013 classification criteria for systemic sclerosis: an American college of rheumatology/European league against rheumatism collaborative initiative. Ann Rheum Dis. 2013 November; 72(11):1747-55.
Clements P J, et al. Skin thickness score in systemic sclerosis: an assessment of interobserver variability in 3 independent studies. J Rheumatol. 1993 November; 20(11):1892-6.

What is claimed is:

1. A method of treating systemic sclerosis in a mammal in need of treatment thereof, comprising administering a therapeutically effective amount of a thromboxane A₂ receptor antagonist or a pharmaceutically acceptable salt thereof to the mammal.

2. The method of claim 1, wherein the mammal is a human patient with a condition selected from the group consisting of dcSSc, lcSSc and SSc-PAH.

3. The method of claim 2, wherein the thromboxane A₂ receptor antagonist is [1S-(1α,2α,3α,4α)]-2-[[3-[4-[(Pentylamino)carbonyl]-2-oxazolyl]-7-oxabicyclo[2.2.1]hept-2-yl]methyl]-benzenepropanoic acid (ifetroban), or a pharmaceutically acceptable salt thereof to the mammal.

4. The method of claim 3, wherein the therapeutically effective amount of ifetroban reduces the rate of formation of sclerotic tissue in the mammal.

5. The method of claim 3, wherein the ifetroban is administered in an amount effective to provide a plasma concentration of the ifetroban of about 1 ng/ml to about 10,000 ng/ml.

6. The method of claim 3, wherein the thromboxane A₂ receptor antagonist is administered in an amount effective to provide a plasma concentration from about 1 ng/ml to about 100,000 ng/ml.

7. The method of claim 3, wherein the therapeutically effective amount is from about 10 mg to about 1000 mg per day.

8. The method of claim 7, wherein the ifetroban is administered orally, intranasally, rectally, vaginally, sublingually, buccally, parenterally, or transdermally.

9. The method of claim 7, wherein the mammal is a human patient and the therapeutically effective amount of ifetroban slows the progression of systemic sclerosis in the patient.

10. The method of claim 7, wherein the mammal is a human patient and the therapeutically effective amount of ifetroban improves the exercise capacity in the patient.

11. The method of claim 7, wherein the mammal is a human patient and the therapeutically effective amount of ifetroban modify the progression of myocardial fibrosis in the patient with dcSSc, lcSSc or SSc-PAH.

12. The method of claim 7, wherein the mammal is a human patient and the therapeutically effective amount of ifetroban improves right ventricular function in the patient.

13. The method of claim 7, wherein the mammal is a human patient and the therapeutically effective amount of ifetroban reduces skin and peripheral vascular disease in the patient compared to placebo as measured by a test selected from the group consisting of digital ulcer imaging, active digital-tip ulcer count, patient reported outcome (VAS), the modified Rodnan skin thickness score, and any combination of the foregoing.

14. The method of claim 7, wherein the mammal is a human patient and the therapeutically effective amount of ifetroban improves pulmonary function.

15. The method of claim 7, wherein the mammal is a human patient and the therapeutically effective amount of ifetroban improves laboratory and physical evidence of inflammation in the patient compared to placebo as measured by serum biomarkers, erythrocyte sedimentation rate, physical examination, and combinations of any of the foregoing.

16. The method of claim 3, wherein the therapeutically effective amount is from about 150 mg to about 350 mg per day.

17. The method of claim 3, wherein the therapeutically effective amount is from about 50 mg to about 500 mg per day.

18. The method of claim 17, wherein the therapeutically effective amount is administered orally.

19. The method of claim 1, wherein the mammal is a human patient with dcSSc, lcSSc or SSc-PAH and the therapeutically effective amount of the thromboxane A₂ receptor antagonist or a pharmaceutically acceptable salt thereof has an action selected from the group consisting of slowing the progression of systemic sclerosis in the human patient as determined by cardiac magnetic resonance imaging (MRI); improving the exercise capacity in the human patient as determined by the six-minute walk test (6MWT); modifying the progression of myocardial fibrosis in the patient as determined by cardiac magnetic resonance imaging (MRI); improving right ventricular function in the patient with dcSSc or SSc-PAH as determined by echocardiography; reducing skin and peripheral vascular disease in the patient with compared to placebo as measured by a test selected from the group consisting of digital ulcer imaging, active digital-tip ulcer count, patient reported outcome (VAS), the modified Rodnan skin thickness score, and any combination thereof; improving quality of life in the patient with dcSSc or SSc-PAH compared to placebo as measured by the patient completed Quality of Life and Scleroderma Health Assessment Questionnaires; improving pulmonary function in patients with dcSSc or SSc-PAH as measured by spirometry and diffusion capacity for carbon monoxide (DL_{CO}); improving laboratory and physical evidence of inflammation in the patient with dcSSc or SSc-PAH compared to placebo as measured by serum biomarkers, erythrocyte sedimentation rate, physical examination, or any combination thereof; and combinations of any of the foregoing.
